(19) United States
(12) Patent Application Publication
(10) Pub. No.: US 2020/0022944 A1
(43) Pub. Date: Jan. 23, 2020

(54) COMPOSITION OR MEDICAL FOOD TO EXTRACT THE DIETARY IRON AND BOOST ITS BIOAVAILABILITY FOR IRON DEFICIENCY (ID) AND ID-ANEMIA

(71) Applicant: TIXUPHARMA DI CARLO GHISALBERTI, Milan (IT)

(72) Inventor: Carlo Angelo GHISALBERTI, Sao Paulo SP (BR)

(21) Appl. No.: 16/488,722

(22) PCT Filed: Feb. 26, 2018
(86) PCT No.: PCT/IB2018/051187

§ 371 Date: Aug. 26, 2019

(30) Foreign Application Priority Data
Feb. 27, 2017 (IT) 102017000021676

(51) Int. Cl. A61K 31/351; A61K 33/34; A61K 38/54; A61K 33/26; A61K 9/00; A23L 33/125; A23L 33/15; A23L 33/16; A23L 29/00; A23L 27/60; A23L 2/52

(52) U.S. Cl. CPC A61K 31/351; A61K 33/34; A61K 38/54; A61K 33/26; A61K 9/0056; A23V 2002/00; A23L 33/15; A23L 33/16; A23L 29/06; A23L 27/60; A23L 2/52; A23L 33/125

(57) ABSTRACT

Pharmaceutical and nutritional composition comprising maltol and/or ethyl maltol for use in the therapy of iron deficiency (ID) and ID-anemia (IDA). The iron-free composition produce an efficient extraction of dietary non-heme iron from ingested food, being designed either as stand-alone maltol and/or etyl maltol-containing oral products, or potentiated by digestive enzymes, hematinic vitamins, copper or other iron-transportpromoters. The inventive compositions prevent gut iron overload typically occurring during therapy by oral iron, which translates into lowered oxidative stress and dysbiosis. The inventive compositions are therefore particularly indicated in IDA subjects intolerant to oral or intravenous (IV) iron.

### FIELD OF THE INVENTION

The present invention relates to “iron free” composition comprising maltol, ethyl maltol or mix thereof for the use in treatment or prevention of iron deficiency and sideropenic anemia.

### BACKGROUND

A prolonged paucity of iron available for erythropoiesis, termed iron deficiency (ID), slowly deteriorates into anemia termed iron deficiency anemia (IDA), aka sideropenic anemia. IDA manifests in rarefied, slightly pale/hypochromic and smaller/microcytic red blood cells (RBCs) when compared to normal RBCs.

The etiologic factors includes bleeding, malabsorption and inflammation (Stein et al. 2016 World J Gastroenterol 2016; 22(35): 7908-7925) often linked to gastrointestinal (GI) or hepatic-renal-cardio disorders. IDA is indeed common in woman of different latitudes due to abundant menstrual bleeding (blood loss) or the increased demand during pregnancy, whilst it's widespread in the overall population of Developing Countries for the concomitance of malnutrition and endemic enteral infections (Lynch SR. J. Nutr. 2011; 141: 763S-768S).

Beside pathologic causes, IDA may be linked to dietary factors. Indeed, a carnivorous diet provides the highly bioavailable heme iron, whilst non-heme iron is poorly absorbed (<10%). Non-hem iron assimilation in turns depends on associated nutritional factors contained in the vegetal foodstuff, that contains both iron-absorption promoters and inhibitors. The formers include ascorbate, citrate, maleate, tartrate and other hydroxyacids. The latters, also named “anti-nutrients’, consist in polyphenols, tannins, oxalate, phosphate, and phytate that hamper assimilation of Fe and other oligoelements (Hazell & Johnson, Br J Nutr. 1987;57:223-233).

The “dialysable iron” test is a common surrogate marker of iron bioavailability (Lakshmi A et al. Int J Food Sci Nutr. 2006; 57(7-8):559-69) used to pre-assess studies on iron-fortification (Argyri et al. Food Chemistry 2011; 127:716-721) or on testing iron bioavailability promoters, e.g. the combination of citric acid, phytase and additional iron in oat-based beverage (Zhang H. et al. Eur. J. Nutr. 2007, 46, 95-102).Whilst a variety of pharmaceutical or nutritional iron, oral formulations are available to treat ID, the new frontier are the GMO plant varieties accumulating high level of iron in tissues.

Yet, in case of supplemental intake, the excess of unabsorbed iron travels into gut wherein it promotes a direct inflammatory/mutagenic action and an aggressive dysbiotic microbiota (Prentice A M et al. Nutrition Reviews 2016; 75(1):49-60). To by-pass the GI route, the intravenous (IV) iron is being increasingly applied into the clinical practice, still it remains a cumbersome and costly therapy.

An alternative “iron-free” approach aims at increasing iron absorption with no need of exogenous supplementation, particularly subjects intolerant to oral iron. Few if any products met the goal, despite new intervention that including an enzymatic pre-treatment by phytase, an enzyme to degrade the potent more iron-sequestring antinutrient, phytate, as in US20100196535 and Nielsen A V F et al (Nutrients 2013, 5, 3074-3098).

An ambitious iron-free approach entails the antagonism of hepcidin, the peptide hormone causing the blockade of iron absorption and circulation. Pieris AG (Germany) is developing Anticalin, a hepcidin-antagonist (W02013087654) with PRS-080 as lead drug for the therapy of inflammatory-driven IDA.

In pursue of more flexible and broader therapeutic tool for the various IDA etiologies—i.e. driven by bleeding, malabsorption and increased demand—we focused on maltol and ethyl maltol. Maltol itself seems to provide ailment in inflammatory bowel disease (IBD,) although the therapeutic dosage (ED₅₀) found with he animal model is met at 140-240 mg/kg bw range (Minaiyan M et al. Int J Prey Med. 2012; 3(Supp11): S162-S169)

Maltol forms a symmetric coordination complex with iron, and was since long studied as iron-donor in IDA (Barrand M A et al. Br J Pharmacol, 1991; 102:408-414/723-729). Recently, Stallmach & Büning. Expert Opin Pharmacother. 2015; 16(18):2859-67 have revamped ‘Ferric maltol’ in ID/IDA and the Rx specialty Ferracru™ was registered with EMA/14567/2016 procedure as anti-IDA therapeutic in IBD after bewer controlled studies.

GB 2128998, EP 0159194, WO 96/41627, WO 09/138761, WO2009138761, WO02/24196, JP 03-067565 disclosed other Ferric maltol complexes or hydrid thereof, either pre-formed or formed in situ, for ID/IDA variants. Moreover, WO2016063228 indicated newer/higher dose regimen of Ferric maltol as efficacious IDA treatment.

Anyhow, none of the examined documents promoted the use of maltol/ethyl maltol for an “iron-free” therapy suited to ID/IDA management, nor disclosed a method to increase the uptake of iron while avoiding its overload in the digestive tract, to avoid its gastrointestinal intolerance and aggression, issues still in need of proper responses.

### SUMMARY

The discovery entails the use of maltol and/or ethyl maltol to enhance the extraction and absorption rate of non-heme iron contained (entrapped) in the current nutrition. Indeed, maltol and ethyl maltol successfully compete with certain antinutrients inhibitor of the absorption of non-hem iron contained in the diet, particularly from plant food, thereby producing a lipophilic, highly biovailable iron complex which undergoing the duodenal uptake.

Conceived for oral administration along main meals, maltol and/or ethyl maltol produce the many-fold increase in bioavailability of dietary non-heme iron. The discovery enables the development of a variety of oral, fast-dissolving solid dose forms (capsules, tablets, powders, etc.) conceived for administration along the main meals. A next generation of medical food such as condiments oil, sauces etc comprising maltol/ethyl maltol is on focus as well.

The inventive compositions enhance the absorption of iron entrapped in the diet, thereby preventing iron overload into large intestine, being particularly suited in subjects intolerant to oral or reluctant to underwent the intravenous (IV) iron. Indeed, the novel ID/IDA therapy aims at avoiding the excess of unabsorbed iron in gut lumen to cause a direct damage or to provoke dysbiosis with unwanted side-effects at the intestinal host-microbiota interface.

Therefore, an inventive object consist in an iron-free digestive composition in dosed form comprising maltol and/or ethyl maltol to prevent or treat of iron deficiency and ID-anemia to be assumed along main meal in the absence of supplementary iron.

Another inventive object consist in a medical food comprising maltol and/or ethyl maltol for the use in prevention or treatment of iron deficiency and ID-anemia.

Another inventive object is a composition or a medical food for the use as defined before which comprises maltol and/or ethyl maltol in combination with at least a digestive enzyme.

A still further inventive object is a composition or a medical food for the use as defined before which comprise maltol and/or ethyl maltol in combination with a cupric compound.

### BRIEF DESCRIPTION OF THE DRAWINGS

FIG. 1 and FIG. 2 show a competition (cell free) test on Fe^{III} of maltol/ethyl maltol versus antinutrients, either as such or upon co-treatment with specific digestive enzymes.

FIG. 3 shows the digestion/extraction test of iron from vegetal food by maltol/ethyl maltol.

FIG. 4 shows the expression of ferritin in Caco2 cells contacted with the digestate if FIG. 3.

FIG. 5 shows the physic-chemical distribution (A), and the expression of ferritin from Caco2 cells (B) in contact with model synthetic iron absorption-promoters and iron sources.

FIG. 6 resume the sequence protocol of the ID-induced animal model.

### DETAILED DESCRIPTION

The term “composition” refers to a broad variety of medicinal products in dosage form including nutritional supplements, pharmaceutical products for human and animals (veterinary scope). It also extends to medicinal food including functional food, food for special medical purpose, and the like.

The inventive composition is characterized by a digestive function, i.e. conceived for oral administration along (in concomitance) with main meals to increased the bioavailability of dietary non-heme iron. Concomitance means administration soon after the start of a meal (preferably), or around ½ hour to 1 hour before, or ½ hour after.

The expression “iron free” or “substantially iron-free” refers to the reduced presence, or marginal or in trace of exogenous/supplementary iron within the composition. It means that the inventive composition may have a limited content of ferrous or ferric atoms, preferably not higher than 1 meq per mmole of maltol o ethyl maltol. Note that an aliquot of Fe could be the impurity in the raw material, or from colorants based on iron oxides (eg tablet coating).

Maltol and ethyl maltol are γ-pyrones homolog, are aroma chemical that confer a toasted note to bakeries, classified as E 636 e E 637 in flavor industry. Maltol for the inventive purpose may be either of natural as it occurs in plant such as larch tree, pine needles, ginseng (Jeong A C et al. Pharmacogn Mag 2015; 11(43): 657-664) and in artifacts like roasted coffee or via chemical origin, the sintethic origin being mandatory for ethyl maltol.

Maltol and ethyl maltol may be presented as such or in hydrated, solvated or in salt form. The salt forms, named “maltolates”, may be produced by acid-base exchange to form addition salt with physiologically acceptable cations. Salts of inorganic base include Na, Li, Mg, ammonium e other cationic ions which may allow the formation of 3:1 maltol-iron complex. Salts of organic base include maltolates of a variety of physiologic or pharmacologic acceptable amines such as isopropopylamine, diethanolamine, triethanolamine, ethanolamine, 2-dimethylaminoethanol, tromethamine, lysine, arginine, glucosamine, and the like.

In preferred embodiment, the inventive composition comprising maltol and/or ethyl maltol is a fast-releasing oral form completed with physiologically acceptable vehicle, excipients and diluent obtainable by a number of ordinary technics.

The digestive compositions according to the invention may be prepared as oral dosage forms by suitable manufacturing method, thus can include customary physiologically acceptable excipients to obtain a palatable fast-dissolving preparation.

The physiologically acceptable excipient can be solid diluents (e.g. Na/Ca carbonates, lactose), disintegrants (e.g. cornstarch), granulating agents, lubricants (e.g. Mg stearate, talc), binders (e.g. starch, gelatine), thickeners (e.g. paraffin, waxes), flavouring agents, colouring agents, wetting agents, emulsifying agents, dispensing agents, preservatives (e.g. parabens, benzoic and sorbic acid), isotonic agents (e.g. sugar, NaCl), fillers, sweeteners, antioxidants, coating materials, buffering agent, and combination thereof.

The composition of invention may be medicinal, i.e. pharmaceutical or nutraceutical preparation obtainable by conventional techniques. Examples of suitable unit dosage forms are tablets, capsules, coated pills, powders in sachets, powder in packets, granules, wafers, as well as liquid preparations. The composition of invention may use solid, semi-solid, or liquid vehicle/carriers to facilitate the delivery of the active ingredient(s).

In an embodiment, the amount of maltol and/or ethyl maltol in the dosed composition varies from 15 to 1500 mg per unit dose, preferably from 25 to 500 mg or more preferably from 50 to 500 mg per unit dose, even more preferably from 165 to 220 mg per unit dose.

Maltol and ethyl maltol may be used separately, or as combination thereof, e.g. at maltol/ethyl maltol ratio from 1:200 to 100:2 w/w in total amount as stated before.

Dose adjustment must be considered in case of low or high, opposite body masses, such as for subjects in pediatric age or in overweight/obese, where the dosed amount of maltol and/or ethyl maltol shall be calculated at 0.1-10 mg/body weight, preferably at 2-5 mg/body weight.

In an embodiment, the inventive composition is a medical food with an anti-anemic (anti-IDA) purpose enriched with maltol and/or etilmaltol. Typical such medical foods are condiment oil and sauces, butter and fat spread, and the like.

Exemplary condiment oil are olive oil, corn oil, sesame oil, sunflower oil, peanuts oil, grape seed oil, soy bean oil, or other edible oils comprising maltol and/or etilmaltol obtained by dissolution at ambient temperature or upon mild heating maltol and/or (preferably) ethyl maltol final amount around 0.1-0.2% w/w or higher.

Exemplary condiments are kectchup, mayonnaise, tartar sauce, tuna fish etc. comprising maltol and/or ethyl maltol solubilized in the oil phase or suspended in the emulsion.

The amount of maltol and/or ethyl maltol shall be opportunely calculated to supply a daily dose from around 15 mg (or less) up to 250 mg or more of maltol/ethyl maltol per serve.

In an embodiment, the composition will further comprise a “digestive enzyme”, expression indicating enzymes having degradative/digestive activity toward nutrients and antinutrients, herein respectively termed “non-classic digestive enzyme” and “classic digestive enzyme”.

Examples of non-classic digestive enzymes, i.e. directed to antinutrients, are phytases and catecholase, which may be of particular utility for vegetarians or vegans ID-subjects.

Suitable phytases comprise 3-phytase (EC 3.1.3.8) and 3-phytase (EC 3.1.3.26) obtained from microorganism or vegetal source, thus include Ronozyme™ NP and Ronozyme™ HiPhos (DSM) obtained from Aspergillus oryzae; Rovabio™ Phy from Penicillicum funiculosum; Quantum™ from Pichia pastoris; Finase™ EC from Trichoderma reesei; Optiphos™ from Pichia pastoris; Ronozyme™ Hiphos from Aspergillus oryzae; Quantum™ Blue from Trichoderma reesei; Natuphos™ from A. niger; Phyzyme™ from S. pombe, etc.

A preferred phytase is 3-phytaseobtained from the fermentation of A. niger, e.g. in dose typically ranging from 500 to 70.000 FTU/dose or more.

The term “cathecol oxidase” (alias catecholase, diphenol oxidase, dopa-oxidase, polyphenol oxidase, pyrocatechol oxidase, tyrosinase) describes various enzymes capable of oxidating the ortho-diphenolic moiety, classified as EC 1.10.3.1. or EC 1.14.18.1 (CAS 9002-10-2).

Examples of classic digestive enzymes, i.e. directed to nutrients, include proteases like pepsin, trypsin or chimotrypsin, either isolated or as mixed enzyme such as pancreatin, which in fact is a blend of trypsin, amilases (amilopsine), lipases (steapsine). These animal proteases may be substituted with functional equivalents obtained from native or genetically modified microorganism, e.g. microbial proteases, acid-stable proteases, or may be substituted with functional equivalents obtained from plants such as papain, bromelain, ficain.

These functional equivalents may be combined with next purified digestive enzymes such as amilases, maltases, lipases, cellulases, lactases, alpha-galactosidases, etc. The inventive composition comprising classic digestive enzymes are particularly suited for ID subjects having a gastric hypo-functionality, e.g. due to the proton pump inhibitor therapy.

In separate embodiment, the composition comprising maltol and/or ethyl maltol are used to prevent or treat a disorder selected from: (a) bleeding-driven ID/IDA; (b) malabsorption-driven ID/IDA; (c) inflammation-driven ID/IDA; or (d) ID/IDA from increased demand; and combination thereof, as better detailed herewith.

(a) Bleeding-driven ID/IDA due to nonvariceal or variceal upper gastrointestinal bleeding, gastric-duodenal ulcer, angiodysplasia and antral vascular ectasia, esophagitis, erosive gastritis and hiatal hernia, inflammatory bowel disease, intestinal failure, intestinal parasitic infection, diverticular disease, restorative proctolectomy, hemorroids, anal fissures and rectal ulcers, gastrointestinal cancer, NSAID-associated blood loss, menorrhagia or metrorrhagia, endometriosis, childbirth, uterine or vaginal cancer, surgery, trauma, or blood donation;

(b) Malabsorption-driven ID/IDA due to H. pylori gastritis, autoimmune gastritis, bariatric surgery, celiac disease, intestinal failure, intestinal parasitic infection, infectious colitis, restorative proctolectomy, malnutrition for high phytate-polyphenol diet; (c) is due to autoimmune gastritis, celiac disease, non-alcoholic fatty liver disease, chronic hepatitis, or liver condition without gastrointestinal bleeding.

(c) Inflammation-driven ID/IDA due to H. pylori gastritis, autoimmune gastritis, bariatric surgery, celiac disease, intestinal failure, intestinal parasitic infection, infectious colitis, restorative proctolectomy, malnutrition for high phytate-polyphenol diet; (c) is due to autoimmune gastritis, celiac disease, non-alcoholic fatty liver disease, chronic hepatitis, or liver condition without gastrointestinal bleeding;

(d) ID/IDA from increased demand due to pregnancy, lactation, childhood, chronic kidney disease (CKD), endurance athletes, patients with coagulation disorders Treatable ID/IDA subject may sufferers from one or more combination of iron deficiency, as highlighted by Hershko & Camaschella (Blood, 2013; 123(3):326-333). Beside humans, also animals, particularly monogastric animals like dog, cat, horse, poultry, etc. will beneficiate the inventive approach.

In an embodiment, the inventive composition further comprises a “hematinic vitamins”. Exemplary hematinic vitamins include vitamin B12 (cyanocobalamin or derivatives thereof) at 2-10 μg/unit dose or higher; and folates (aka vitamin M, vitamin B9, folacin and derivatives) at 100-1000 μg/unit dose or higher.

In an embodiment, the inventive composition further comprise a polyacid to marginally increase the capacity of maltol or ethyl maltol to recover dietary iron. Exemplary polyacids include: ascorbate, citrate, maleate, tartrate, succinate, oxalacetate, ketoglutarate, isocitrate, and polyphosphate, in amount from 50 to 5000 mg/unit dose.

In an embodiment, the inventive composition further comprise a cupric compound to ameliorate the homeostasis of iron introduced as maltol/ethyl maltol complex. Exemplary cupric compounds include cupric carbonate, cupric citrate, cupric gluconate, cupric solfate, cupric-lysine complex, cupric pidolate, etc. in amount from 0.1 mg to 10 mg/unit dose.

Recommended posology in preventive or curative anti-anemic therapy by the inventive compositions consist in 2-3 daily administrations in concomitance with main course for at least a month, preferably 2-3 months or the time need to restore or maintain an hemoglobin at around 14 g/dl in man and 12 g/dl in women (11 mg/dl for pregnant) and/or the ferritin level at normal values: 60-140m/dl.

The inventive compositions are essentially iron-free, hence particularly indicated in IDA subjects intolerant to oral or IV iron. The advantage over iron supplementation is to prevent iron overload in lower intestine, which inter alia translates into lower dysbiotic disorders.

### EXAMPLES

### Example 1—Cell-Free In Vitro Competition Test

The method evaluates the competitive affinity for Fe(III) of maltol/ethyl maltol versus antinutrients, i.e. the iron sequestering agents occurring in food, in a 3-step method which adopts a sequential pH 6/2.5/6.5 mimic the pHs occurring in stomach at fast, during gastro digestion and in duodenum, respectively.

Stock solution at 10 mM conc. were prepared by dissolving in water the following reagents:

(a) antinutrient series: EDTA (EDTA=negative control), ellagic acid (ELL), gallic acid (GAL), hesperidin (HES), naringin (NAR), monosodium oxalate (OXA), disodium phosphate (PHO), quercetin (QUE), rutin (RUT), tannic acid (TAN), phytic acid sodium salt (PHY); wherein the cathecolic polyphenols (CPP) are presented as pooled

(b) maltol (MAL) and ethyl maltol (EMAL);

(c) Fe(III) and Ca sources: ferric ammonium citrate (FAC) and CaCl₂ 10H₂O, respectively.

In brief, in 15-ml tubes were combined 100 μl of 10 mM FAC (1 μmol); 1 ml of 10 mM CaCl₂ (10 μmol); 1 ml of 10 mM 3-hydroxy-4-pyrone (10 μmol); 1 ml of each antinutrient at 10 mM (10 μmol); and 2 ml of water to 5 ml final volume. No antinutrients were introduced in the positive control. Tubes were oscillated at 30 rpm for 2.5 hours, of which: 1 hour at initial pH 6-6.5; 1 hour at pH 2-2.5 (by 50 μl 1N HCl), and final 30 min. at pH around 6.5 (by 50 μl 1N NaOH). The tubes were then added with 2 ml di CH₂Cl₂ and shacked for 2 min. Aliquots (200 μl) of organic phase were placed in glass vials and dried on warm air stream. Residues were taken up with 2 ml water and read at 405 nm. Absorbance calibration: r=0.30 and linear correlation within the 40-4 μM range (r2=1; r2=1.97877 on 0-intercept).

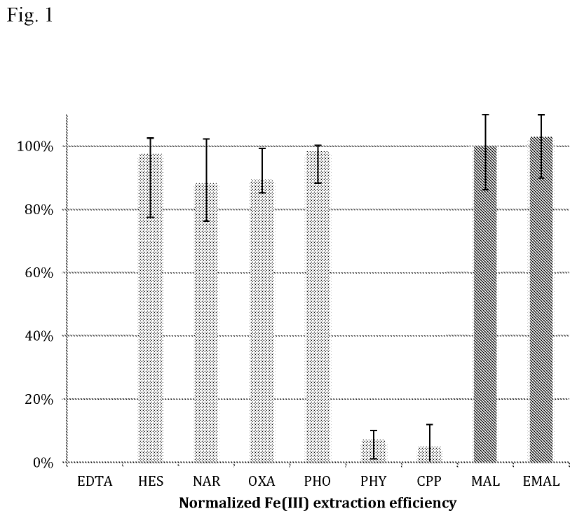

Data plotted in FIG. 1 shows that maltol or ethyl maltol compete with citrate (ref standard of 100% recovery) and antinutrients, viz. oxalates (89% recovery), phosphate (98% recovery), and non-cathecolic polyphenols (avg. 95% recovery) thereby forming 1:3 Fe-ligand complexes with high biovailability. NB, the complexes are easily and quantitatively assessed due to the their solubility in CH₂Cl₂, which allow neat separation from the aqueous solution.

### Example 2—Cell-Free Competition Test with Enzymatic Pretreatment

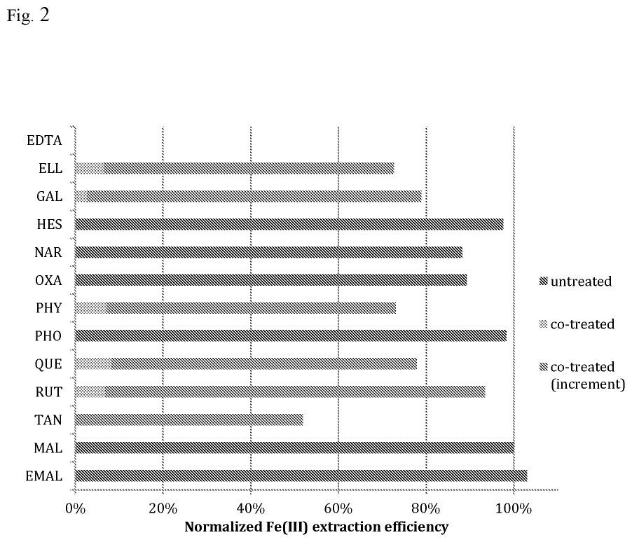

As data from example 1 shows a limited efficiency in the iron competition versus phytate and catecholic polyphenols, resistant antinutrients were subject to co-treatment with specific degrading enzyme. This entailed mixing 5 mg of polyphenol oxidase (Sigma-Aldrich T3824; 1000 U/mg) at pH 6.5 on ELL, GAL, RUT, and TAN; or 50 mFTU (Ronozyme HiPhos) at pH 4,5 for phytic acid sodium salt. Specimen underwent the same procedure as in Example 1, affording the results plotted in FIG. 2 showing cumulative pre- or co-treatment ability of maltol/ethyl maltol to compete with antinutrients in the race for ferric ions.

### Example 3—In Vitro Digestion Model

The method assessed the extraction of dietary non-heme iron by maltol or ethyl maltol in simulated digestion of vegetal foods, with/without digestive enzymes. In brief, samples (10 g) of vegetable foods, viz. canned soybean, red beans and peas were smashed in mortar with 5 ml water. Specimen were combined in 50 ml tubes supplemented with HCl solution (pH 2.5) of mix of classic digestive enzymes consisting in: 2 mg pepsin 1:3000; 0.5 mg lipase 200 FIP/g; and bile enzymes, i.e. 2.5 mg bovine bile extract; 1.25 pancreatin extract 4:1 in “gastroactive” model (“GA”). A parallel test named “gastropassive” use non enzymes. Tube were added with 1 ml maltol/ethyl maltol 10 mM, or water in control, and underwent 2 hours of slow motion (60 o/min) at controlled temperature. The pH was finally adjusted to 6-7 (1N NaOH) and the tubes remained on oscillation for further 30 min.

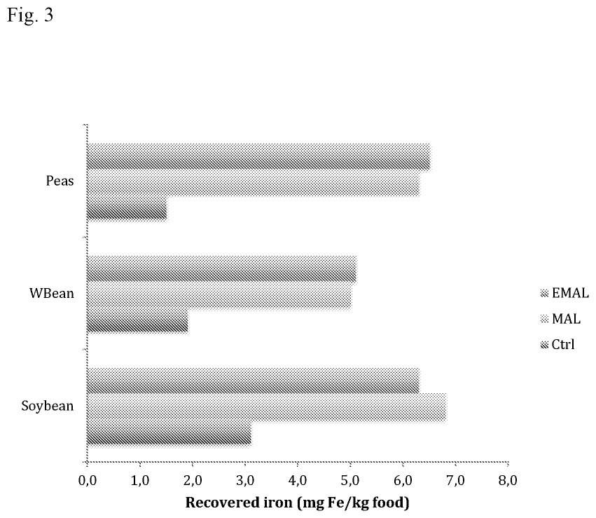

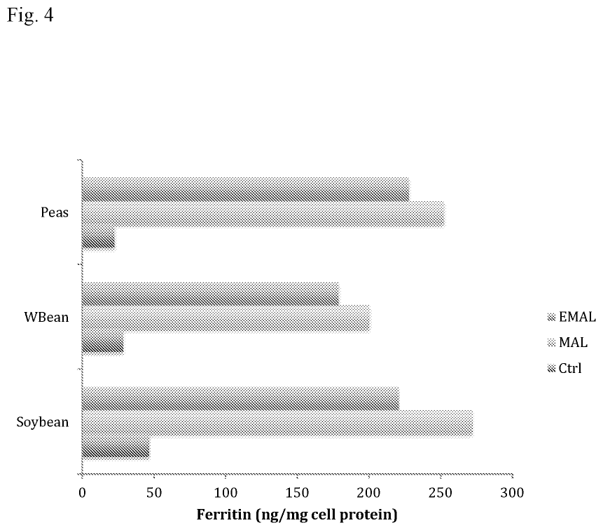

The digests were collected, filtered at 100 μm mesh, diluted and read at 405 nm. Results expressed as % recovery of Fe calculated against a calibration curve) are plotted in FIG. 3.

In this test maltol and ethyl maltol increased the yield of extracted iron from vegetal food, meaning a significant increase in dietary iron bioavailability.

Noteworthy, yields of GA model slightly surpassed by about 10% those obtained in gastropassive (GP) condition suggesting an optimal combination of maltol/ethyl maltol with digestive enzymes in subjects with defective gastro-functionality, e.g. in therapy with PPI.

### Examples 4, 5—Caco-2 Monolayer Absorption

This study investigates the boost in iron bioavailability by maltol/ethyl maltol compared to ascorbate, a known absorption promoter, in relation to specific iron sources.

Caco-2 cells cultivated and used at passages 24-50 in culture medium, serum-free MEM, were exposed to test substances in culture medium.

In example 4, aliquots obtained from the digestates of Example 3 have been seeded on the apical layer of CaCo-2 cells.

In example 4, pure chemicals ere seeded on Caco-2 cells starting from the following stock solution prepared in MEM (Minimum Essential Medium, pH 7.4):

(a) maltol (MAL) and ethyl maltol (EMAL) at 10 mM concentration;

(b) iron-absorption promoter: ascorbic acid (AA) and citric acid (CA), both at 20 mM conc;

(c) iron sources: FAC (Fe^{III}) and ferrous sulfate (FS, Fe^{III}) at 1:10 eq/eq with respect to AA, at 1:20 eq/eq with respect to CA, and at 1:5 eq/eq with respect to MAL.

The final Fe concentration was fixed 200 μM with the other molar ratio as listed above, while the solution were then neutralized to pH 6-7.

One hour contact at 37° C. was used for similarity of duodenal contact time. After incubation 1 h the medium was aspirated, cell monolayer washed 3× PBS-EDTA and fresh MEM added. Cells were re-incubated for 23 h to allow ferritin formation, lysed with 400 μl of MPER® lysis buffer, lysate collected, centrifuged for 5′ at 16,000×g, and supernatant collected for analysis. The solutions were filter-sterilized (0.2 μm syringe filter) prior dilution in the culture medium.

Total iron was determined by inductively-coupled plasma optical emission spectrometry, where both ICP-OES standards and samples were diluted in 0.5% HNO₃ in 0-1000 ppb range. Physical classification of iron was assessed after centrifugation (10,000×g, 5 min.), the supernatant was ultrafiltrated with 3kDa MWCO filter 10,000×g, 10 min. Phase distribution was calculated as: iron microparticulate (total Fe−Fe in supernatant), iron nanoparticulate (Fe ultrafiltrate−Fe in supernatant) and soluble iron (Fe ultrafiltrate/total Fe).

Ferritin content of Caco-2 cells was determined by an ELISA kit, results corrected for the baseline values and expressed in relation to total cell protein in ng ferritin/mg cell protein.

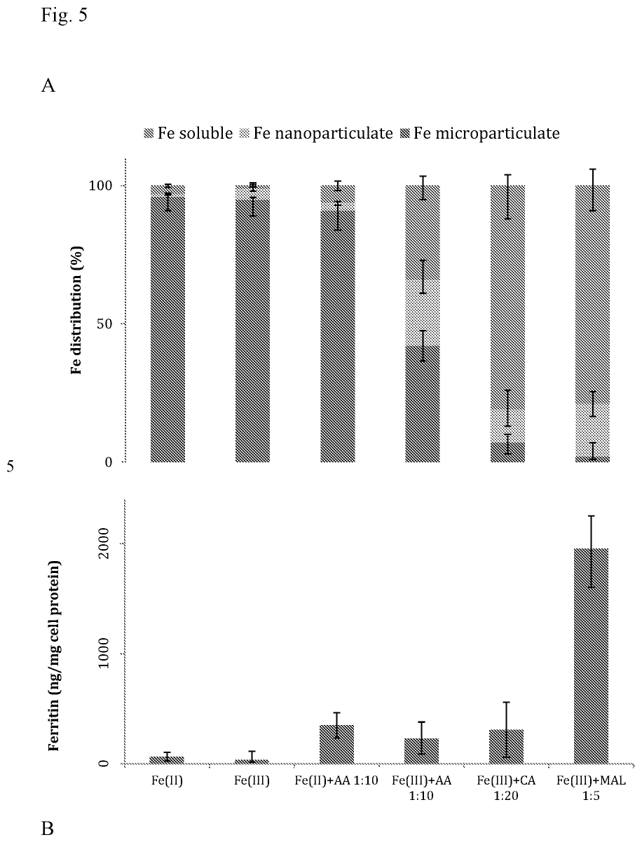

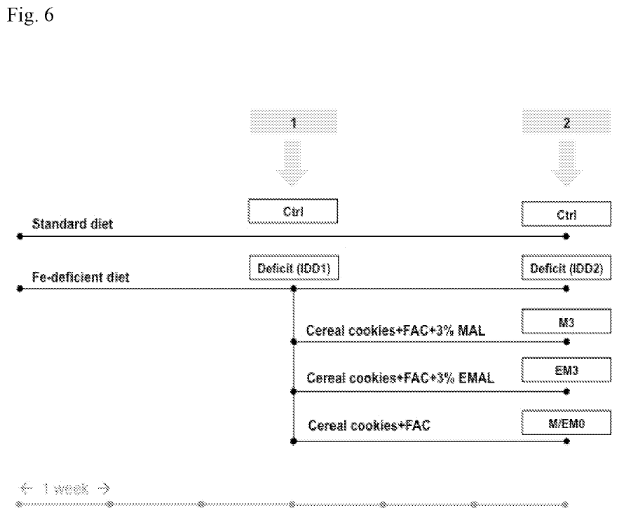

FIG. 5B show that iron(II) and Iron(III) salt are poorly soluble. Instead, ascorbate in the presence of Fe(II) sulfate partially prevented the issue, while citric acid produced high solubility. In contrast, maltol retain the Fe(III) solubility at supplemental levels, detectable by almost no precipitate and low nanoprecipitate in suspension.

The formation of ferritin, an indirect assessment of iron availability, depicted in FIG. 5B, maltol and ethyl maltol produced soluble iron utilized by enteric cells for ferritin synthesis. Thus, enhanced iron solubility prompted by positively correlates with the stimulation of ferritin expression, whose enhanced expression also indicates a high efficient uptake system for the in-situ formed Maltol-iron complex.

### Example 6—Murine Model of Anemic Therapy

This in vivo murine study sized the capacity of maltol/ethyl maltol compounded into food to enhance iron uptake in rodents conducted to anemia by iron starvation. For that purpose, 6-wk old female Wistar rats were fed for 28 days a iron-deficient diet (Fe:12 mg/kg), labeled IDD1. A control group received standard diet (Fe:45 mg/kg) (Ctrl1) along the entire experiment.

In the second stage, IDD1 rats were randomized for 26 days test diets conceived as follows: the M3 and EM3 groups received cookies with 3% of maltol or 3% ethyl maltol, respectively. The M/EMO group was fed with cookies without maltol/ethyl maltol; while the IDD2 group was maintained at Fe-deficient diet. Cereal cookies, the base diet in the M3, EM3 and M/EMO groups contained ferric ammonium citrate (FAC) at 45-48 mg Fe/kg meal. Cookies were produced from corn flour and vegetal fat, and backed at 180° C. for 15 minM, whereas in M3 and EM3 groups a portion (3%) of corn flour was replaced with maltol and ethyl maltol.

Animals were kept in room with 12 h light/dark cycle, temperature of 24° C. 35 6° C., and drinking water ad libitum. At the start and at the end of any stage, tail blood was collected for hematologic analysis.

Serum iron was measured at 623 nm, haemoglobin content (Hb) total iron binding capacity (TIBC), erythrocytes, haemoglobin (Hb), hematocrit (Ht), mean corpuscular volume (MCV), mean corpuscular haemoglobin (MCH), and MCH concentration (MCHC) assessed according to Regula et al. Ann Agric Environ Med 2016;23(2):310-4. Iron transferrin saturation (TSAT) was calculated by the formula: TSAT=(Fe/TIBC)×10. Results are reported in Table 1.

TABLE I

| | First stage | | Second stage | | | | |
|---|---|---|---|---|---|---|---|
| | IDD1 | Ctrl | M3 | EM3 | E/EM0 | IDD2 | Ctrl |
| TSAT | 6.51 ± 1.40 | 20.4 ± 1.68 | 23.7 ± 2.75 | 24.5 ± 6.22 | 12.3 ± 4.86 | 12.3 ± 4.86 | 21.5 ± 3.10 |
| RBC | 7.86 ± 0.42 | 7.59 ± 0.45 | 5.43 ± 1.71 | 5.26 ± 2.45 | 6.71 ± 1.50 | 9.18 ± 2.01 | 8.40 ± 3.64 |
| (T/L) | | | | | | | |
| Hb | 7.28 ± 0.39 | 8.84 ± 0.40 | 8.34 ± 1.92 | 8.32 ± 2.76 | 7.53 ± 0.98 | 6.36 ± 1.35 | 9.26 ± 2.07 |
| (mmol/L) | | | | | | | |
| Ht (1/L) | 0.36 ± 0.02 | 0.41 ± 0.01 | 0.36 ± 0.02 | 0.37 ± 0.03 | 0.34 ± 0.01 | 0.33 ± 0.01 | 0.43 ± 0.04 |
| MCV (fL) | 46.8 ± 0.88 | 54.9 ± 0.86 | 52.9 ± 2.30 | 54.8 ± 1.37 | 52.4 ± 1.40 | 37.8 ± 0.96 | 52.4 ± 1.78 |
| MCH | 0.09 ± 0.03 | 1.15 ± 0.05 | 1.60 ± 0.19 | 1.51 ± 0.17 | 1.19 ± 0.22 | 0.93 ± 0.17 | 1.11 ± 0.08 |
| (fmoL) | | | | | | | |
| MCHC | 19.1 ± 0.34 | 20.0 ± 1.68 | 29.4 ± 1.53 | 27.1 ± 2.05 | 22.4 ± 1.38 | 25.1 ± 1.84 | 20.3 ± 1.13 |
| (mmol/L) | | | | | | | |
| Fe | 13.1 ± 2.34 | 17.6 ± 1.26 | 18.7 ± 1.38 | 18.4 ± 2.03 | 15.1 ± 3.12 | 11.3 ± 1.92 | 19.4 ± 3.61 |
| (mmol/L) | | | | | | | |

In conclusion, feeding Wistar rats for 26 days with an ID diet and then supplemented with a normalized-iron vegetal diet comprising maltol or ethyl maltol resulted in faster recovery of iron and hematologic levels. The effect is manifested as increased rate of these parameters: iron transferrin saturation (TSAT), hemoglobin (Hb), mean corpuscular volume (MCV), mean corpuscular hemoglobin (MCH) and mean corpuscular hemoglobin concentration (MCHC) values, that increased considerably compared to both controls.

### Formulation Examples

The composition hereafter are non-limitative examples of dosed forms suitable for the inventive purpose, i.e. Iron-absorption promoter compositions (IAPC) to be consumed along main meals to boost (non-heme) iron extraction/uptake from foodstuff.

While several dosed composition can be produced under standard manufacturing methods, a variety of customary excipients (omitted) may be used to complete the formulation.

### Example 7—IAPC Tablets

| Ingredient | Amount |
|---|---|
| Maltol | 225 mg |

### Example 8—IAPC Tablets

| Ingredient | Amount |
|---|---|
| Ethyl maltol | 200 mg |
| Maltol | 150 mg |
| Choline HCl | 250 mg |

### Example 9—IAPC Tablets with Selected Hematinic Vitamins

| Ingredient | Amount | |
|---|---|---|
| Maltol | 165 | mg |
| Copper gluconate | 3.74 | mg |
| Folic acid | 0.1916 | mg |
| Vitamin B12 0.1% | 6.5 | mg |

### Example 10—IAPC Sachets with Broad Range Vitamins

| Ingredient | Amount per sachet | |
|---|---|---|
| Vitamin A | 300 μg RE | |
| Vitamin D | 5 | μg |
| Vitamin E | 5 | mg |
| Vitamin C | 30 | mg |
| Thiamin (vitamin B1) | 0.5 | mg |
| Riboflavin (vitamin B2) | 0.5 | mg |
| Niacin (vitamin B3) | 6 | mg |
| Vitamin B6 (pyridoxine) | 0.5 | mg |
| Vitamin B12 (cobalamin) | 0.9 | μg |
| Zinc | 5 | mg |
| Copper (as citrate) | 0.56 | mg |
| Selenium | 17 | μg |
| Iodine | 90 | μg |

### Example 11—IAPC Capsule with Classic Digestive Enzymes (Vegans)

| Ingredient | Amount | |
|---|---|---|
| Ethyl maltol | 120 | mg |
| Protease | 6312 | HUT |
| Acid stable protease | 55 | SAPU |
| Amilase | 1375 | DU |
| Lipase | 412 | FIP |
| Cellulase | 250 | CU |
| Lactase (non-milk derived) | 100 | ALU |
| Papain | 3300 | PU |
| Bromelin | 2750 | PU |
| Alfa-galactosidase | 22 | GaIU |

### Example 12—IAPC Hard Capsule with Classic Digestive Enzymes (Animal-Sourced)

| Ingredient | Amount | |
|---|---|---|
| Maltol | 70 | mg |
| Pancreatin ox bile extract | 200 | mg |
| Pepsin 1:3000 | 200 | mg |
| Pancreatin, extract 4:1 | 125 | mg |
| 3-Phytases from A. niger (525 ≈ FTU) | 5 | mg |

Gelatin/glycerin 0.4:1 w/w hard capsule.

### Example 13—IAPC Syrup

| Ingredient | Amount in 100 ml | |
|---|---|---|
| Maltol | 1.0 | g |
| Sucrose-Xylitol 1:2 | 60 | g |
| Caramel (E150) | qb | |
| Benzoic acid (E210) | 1 | g |
| Ethyl alcohol | 4 | g |

| | |
|---|---|
| Water | qb to 100 |

The syrup was loaded into bottle equipped with a graduated cup (5/10/15 ml marks).

### Examples 14-18—Condiment Oils for Iron Assimilation (Medical Food)

Maltol and ethyl maltol were added under mixing/heating on dietetic oil and fats as set forth in Table 2, where: 14: olive oil; 15: sunflower oil; 16: butter; 17: ghee; 18: margarine.

TABLE 2

| Values in 100 g | Ex. 14 | Ex. 15 | Ex. 16 | Ex. 17 | Ex. 18 |
|---|---|---|---|---|---|
| Calories (Kcal) | 874 | 884 | 717 | 746 | 717 |
| Fats (g) | 99.2 | 99.7 | 81 | 91.6 | 81 |
| of whom saturated (g) | 15.2 | 15.1 | 51 | 93.4 | 15 |
| Cholesterol (mg) | | | 215 | 243 | 0 |
| Carbohydrate (g) | | | 0.1 | <0.1 | 0.7 |
| Protein (g) | | | 0.9 | <0.1 | 0.2 |
| Sodium (mg) | | | 11 | <1 | 2 |
| Potassium (mg) | | | 24 | <1 | 18 |
| Maltol (mg) | 50 | | 125 | 100 | 200 |
| Ethyl maltol (mg) | 400 | 500 | 400 | 350 | 200 |
| total per serve (mg)* | 22.5 | 75 | 78.75 | 67.5 | 60 |

*calculated on 15 ml serving.

### Examples 19-22: Condiment Sauces for Iron Assimilation (Medical Food)

Sauces from Calvé™-Unilever (23-25) and Cirio (26) were added with maltol/ethyl maltol producing sauces as of Table 3, where: 19: mayonnaise; 20: tartar sauce; 21: tuna sauce 22: tomato sauce.

TABLE 3

| Values in 100 g | Ex. 19 | Ex. 20 | Ex. 21 | Ex. 22 |
|---|---|---|---|---|
| Calories (Kcal) | 610 | 430 | 660 | 56 |
| Fats (g) | 68 | 42 | 71 | 1.4 |
| of whom saturated (g) | 8 | 5 | 9 | 0.3 |
| Carbohydrate (g) | 4 | 10 | <0.5 | 8.3 |
| Of whom sugars (g) | 1 | 6 | <0.5 | 5.6 |
| Proteins (g) | 0.8 | 1 | 3 | 1.5 |
| Salt (g) | 1.1 | 2.6 | 1.6 | 0-6 |
| Maltol (mg) | 170 | 100 | 140 | 100 |
| Ethyl maltol (mg) | 170 | 200 | 100 | 200 |
| total per serve (mg) | 51* | 30** | 36* | 45** |

*serving calculated on 15 g: ** ; 10 g; *** .

### Examples 23-26—Beverages for Iron Assimilation (Medical Food)

Beverage from brand Santal™ (Parmalat) were admixed with maltol by turbo-mixing to produce dietetic beverages as set forth in Table 4, wherein: 23: red orange; 24: modified Fructalact™; 25: pink grapefruit; 26: pineapple.

TABLE 4

| Values in 100 g | Ex. 23 | Ex. 24 | Ex. 25 | Ex. 26 |
|---|---|---|---|---|
| Calories (Kcal) | 46 | 46 | 40 | 47 |
| Carbohydrates (sugars) (g) | 10.5 | 9.9 | 10 | 11 |
| Proteins (g) | 0.1 | 0.8 | <0.1 | 0.3 |
| Sodium (mg) | 5 | 30 | <5 | <1 |
| Maltol (mg) per 100 ml* | 65 | 120 | 90 | 150 |
| (typical serving) | | | | |

### Example 27—Case Series

Current clinical studies on selected ID patients are in progress. The results will become available after the publication of the present document.

### Example 28—IAPC Effervescent Tablets

| Ingredient | Amount |
|---|---|
| Citric acid/Sodium bicarbonate 2:4 w/w | 400 mg |
| Maltol | 225 mg |

1. Method of treating iron deficiency (ID) or ID-anaemia with maltol or ethyl maltol, said method comprising:
a) orally administering a therapeutically effective amount of said maltol or said ethyl maltol along with main meals to improve bioavailability and absorption of the dietary non-heme iron;
b) orally administering of said maltol or said ethyl maltol in substantial absence of supplementary iron;
c) orally administering said maltol or said ethyl maltol oral an iron deficient subject intolerant/reactive to intestinal iron overload.

2. A method of treating or preventing iron deficient anemia in subjects in need thereof, said method comprising administering maltol and/or ethyl maltol in substantial absence of supplemental iron (iron free) to said subjects.

3. The method according to claim 2 comprising administering said maltol and/or said ethyl maltol in amount higher than 20 mg/dose.

4. The method according to claim 3, wherein said maltol, said ethyl maltol or mix thereof are in amount from 50 to 500 mg/dose.

5. The method according to claim 2, comprising administering from 50 to 250 mg per serving of maltol, ethyl maltol or mixture thereof.

6. The method according to claim 2 to further comprising administering a classic or non-classic digestive enzyme.

7. The method according to claim 2 further comprising administering a cupric compound.

8. The method according to claim 2 further comprising administering at least a hematinic vitamin.

9. The method according to claim 2, wherein said iron deficiency or ID-anaemia is driven by gastrointestinal or genital bleeding, intestinal malabsorption, ulcerative or infective inflammation, use of erythropoiesis-stimulating agents, chronic kidney disease, cardiac insufficiency, and combination thereof.

10. The method according to claim 2, wherein said subjects suffering from iron deficiency or ID-anaemia are cologic patients.

11. The method according to claim 2 wherein said subjects comprise women in fertile age with increased demand due to consistent urogenital bleeding (metrorrhagia, endometriosis, etc.), pregnancy or lactation.

12. The method according to claim 2, wherein said subjects suffer of chronic kidney disease (CKD), congestive heart failure (CHR), inflammatory bowel disease (IBD), or combination thereof.

13. The method according to claim 2 comprising orally administering said maltol or said ethyl maltol oral to an iron deficient subject intolerant/reactive to intestinal iron overload.

14. Composition comprising maltol and/or ethyl maltol in amount higher than 20 mg/dose, an amount of iron that is not higher than 1 meq per mmole of maltol and/or ethyl maltol and physiologically acceptable vehicles, excipients and diluents.

15. Composition according to claim 14, wherein maltol, ethyl maltol or mix thereof are in amount from 50 to 500 mg/dose.

16. Composition according to claim 15, comprising inorganic salts of maltol or ethylmaltol, said salts comprising Na, Li, Mg and ammonium.

17. Composition according to claim 15, comprising maltol or ethylmaltol salts of an organic base selected from pharmacologic acceptable amines selected from the group consisting of isopropopylamine, diethanolamine, triethanolamine, ethanolamine, 2-dimethylaminoethanol, tromethamine, lysine, arginine, glucosamine, and mixtures thereof.

18. Composition according to claim 15 further comprising a digestive enzyme.

19. Composition according to claim 15 further comprising a cupric compound and/or a hematinic vitamin.

20. Composition according to claim 15 comprising a medical food selected from condiment oil and sauces, butter and fat spread.
